(19) United States
(12) Patent Application Publication
(10) Pub. No.: US 2019/0106698 A1
(43) Pub. Date: Apr. 11, 2019

(54) METHOD FOR APTAMER PAIR SELECTION

(71) Applicant: PROXIMITY BIOSCIENCES, LLC, Auburn, AL (US)

(72) Inventor: Joonyul KIM, Lexington, SC (US)

(73) Assignee: PROXIMITY BIOSCIENCES, LLC, Auburn, AL (US)

(21) Appl. No.: 16/211,616

(22) Filed: Dec. 6, 2018

Related U.S. Application Data
(63) Continuation-in-part of application No. PCT/US2017/037856, filed on Jun. 16, 2017.
(60) Provisional application No. 62/351,890, filed on Jun. 17, 2016.

(51) Int. Cl. C12N 15/115; C40B 50/06; C12Q 1/686; C12Q 1/6806; C40B 40/08

(52) U.S. Cl. CPC C12N 15/115; C40B 50/06; C40B 40/08; C12Q 1/6806; C12Q 1/686

(57) ABSTRACT

Methods for selecting single or multiple aptamer pairs against target molecules in free solution have been developed. These methods utilize novel cooperative evolution approaches to select aptamer pairs against one or more targets, in which the pairing of one or more aptamers upon target binding triggers aptamer amplifiability. In this manner, the enrichment of aptamer ligands through one or multiple rounds of the selection process is based predominantly upon target-driven close proximity of aptamers in free solution. Target binding and enrichment are coupled using either positive or negative selection methods. These techniques should be generally applicable to many different types of target molecules, providing alternatives to antibodies, drugs, or other binding molecules for analytical, preparative, and therapeutic purposes.

### CROSS-REFERENCE TO RELATED APPLICATION

This application is a continuation-in-part of PCT/US2017/037856, filed Jun. 16, 2017, which claims the benefit of priority from U.S. Provisional Application Ser. No. 62/351,890, filed Jun. 17, 2016, the contents of each of which are incorporated herein by reference.

### FIELD OF INVENTION

The present invention relates to methods, reagents, and kits for obtaining paired oligonucleotide aptamers, selective for a target of interest, starting from libraries of randomized oligonucleotides.

### BACKGROUND OF THE INVENTION

Aptamers are small artificial ligands, including single stranded DNA, RNA or polypeptide molecules which are capable of binding to specific target moieties of interest with high affinity. Aptamers have been regarded as potential alternatives to antibodies for use as diagnostic and/or therapeutic purposes for about twenty-five years. Aptamers are generally obtained by screening a random library of candidate oligonucleotides by an affinity based partitioning against the target of interest. Aptamers have high structural stability over a wide range of pH and temperatures making them ideal reagents for a broad spectrum of in-vitro, ex-vivo, and in-vivo applications.

In 1990, Tuerk et al., 1990 (Science 249, 505-510, doi. 10 1126/science.2200121) and Ellington 1990 (Nature 346, 818-822, doi:10.1038/346818a0) developed an in-vitro method that mimics the evolutionary process. The process was called Systematic Evolution of

Ligands by Exponential Enrichment (“SELEX”). The SELEX process exploits fundamental concepts of evolution, utilizing variation, selection, and replication to achieve high target affinity and specificity from a starting pool of nucleic acid molecules (i.e., oligonucleotides). In general, for selection of nucleic acid (oligonucleotide DNA or RNA) aptamers, variation is achieved by synthesizing a library of short oligonucleotides (about 10¹⁴ different sequences), ranging in size from about 20 to about 100 nucleotides. Each oligonucleotide comprises an internal random region flanked by primer regions for subsequent amplification by suitable amplification reaction, e.g., the polymerase chain reaction (PCR) for DNA, or reverse transcription polymerase chain reaction (RT-PCR) for RNA. Due to the large number of unique sequences in the library, the probability of at least some aptamer molecules to bind the target with specificity and affinity is high. Next, selection is achieved by affinity based partitioning (e.g., incubating the nucleic acid pool with target molecules immobilized onto beads, then washing away the non-binding sequences). Replication is achieved by amplifying the bound oligonucleotides using PCR or some other amplification method. The pool of oligonucleotides obtained from the round of SELEX (i.e. selection followed by amplification) is then used as an input for the next round of SELEX until a set of oligonucleotides (i.e. nucleic acid aptamers), are enriched, which bind tightly and specifically to the target molecule(s). Recent technical advances in the preparation of random libraries and in affinity-based partitioning made aptamers have equivalent affinities to antibodies.

This technology has been applied to select DNA or RNA aptamers against inorganic components, small organic molecules (Ellington, et al., 1990, Nature 346, 818-822, doi:10.1038/346818a0), nucleotides (Sassanfar 1993, Nature 5;364(6437):550-3), cofactors (Lorsch 1994, Biochemistry, 33(4):973-82), nucleic acids (Boiziau, et al., 1999, J. Biol Chem. 30;274(18):12730-7), amino acids (Majerfeld et al., 2005 J Mol Evol. 61(2):226-35), carbohydrates (Jeong et al., 2001, Biochem Biophys Res Commun. 16;281(1):237-43), antibiotics (Wang 1996, Biochemistry, 35(38):12338-46.), peptides (Ylera et al., 2002, Biochem Biophys Res Commun. 290(5):1583-8), proteins (Tuerk, 1990 as above), and even complex structures such as cells (Shangguan et al., 2006 Proc Natl Acad Sci USA. 103(32):11838-43).

The advantages of aptamers over antibodies are: ease of in vitro synthesis, flexible modification, broad target ranges, reusability, and high thermal/chemical stability. Non-immunogenicity and the availability of antidote add the value of aptamers as therapeutic drugs.

Generally, bioassay requires a pair of ligands to achieve high sensitivity and specificity in detection. Also, the linked pair would be a new ligand enhancing its affinity and specificity. In this respect, antibodies outcompete to aptamers until now. A pair of antibodies is fairly easily obtained because of their pre-designed antigenic binding sites before the production. In contrast, it is difficult to obtain a set of aptamers having different binding sites of a target due to the fundamental limitation of a traditional SELEX scheme. Aptamers have been enriched from a randomized library, without prior knowledge of the binding site(s), so it is impossible to assign the binding site to the individual aptamer in the pool using currently available methodologies.

In the conventional aptamer selection scheme, the pair selection from aptamer-enriched pools is regarded as a post-selection step after identification of individual aptamers. To date, there have been only three successful platforms for aptamer pair selection. All of these are based on the “brute-force” strategies to either (1) select a single aptamer, block its binding site, then start over and select another single aptamer (Ochsner, et al., 2014, BioTechniques 56, 125-133, doi:10.2144/000114134 and Csordas, et al., 2016, Anal. Chem 88, 10842-10847, doi: 10.1021/acs.analchem.6b03450) or (2) identify individual aptamers in a pool or library and then conduct pairwise testing for simultaneous binding with the built-up aptamer matrix (Cho, M. et al., 2015, Analytical chemistry 87, 821-828, doi:10.1021/ac504076k). The former approach is time-consuming and the latter, “shot-gun” approach, has to rely on the size of aptamer matrix to be tested for its success, which is correlated with the expense.

This decoupled approach has resulted in a prohibitively high economic cost and a low efficiency in finding aptamer pairs, therefore, there remains a long-standing need for improvements in speed and efficiency for selection of aptamer pairs. The present invention hereby provides a highly efficient novel method for target-driven selection of RNA aptamer pairs, wherein RNA aptamers are selected simultaneously as pairs capable of selectively binding to the same target of interest.

### SUMMARY OF THE INVENTION

The present invention provides an aptamer pair selection method, reagents and kits for enriching paired oligonucleotide aptamers. The aptamer pair selection methodology according to the present invention selects pairs of aptamers from random pools against a free-solution target by allowing only pairs of oligonucleotides bound to the target of interest to survive during the selection process. Through the present invention, aptamers in a pair can be selected simultaneously, alleviating many of the current problems of high expense, low efficiency, and tedious workflows. The homogeneous nature of the present invention offers multiplexability, scalability, robustness and ease of monitoring at every round of selection.

Broadly, the invention provides a method for isolating pairs of oligonucleotide aptamers for selective binding to a target of interest, the method comprising:

(a) preparing two libraries of sequence randomized oligonucleotides;

(b) independently screening each library of (a) by affinity based partitioning against the target of interest, to obtain respective A and B pools of oligonucleotides enriched with oligonucleotides that bind to the target of interest;

(c) incubating the pool A and pool B oligonucleotides with the target of interest, and a connector oligonucleotide, in order to form a four part complex of two oligonucleotides, the connector and the target of interest;

(d) adding a ligating enzyme to the product of (c) to ligate the oligonucleotides of the complex to form a ligated oligonucleotide; and

(e) amplifying the ligated oligonucleotide of (d) by a polymerase chain reaction (PCR) or by a reverse transcription polymerase chain reaction (RT-PCR) to produce a DNA oligonucleotide encoding two oligonucleotide aptamers.

The method further comprises subjecting the oligonucleotide pairs to one or more additional cycles of enrichment by repeating steps (c) through (e) until 13-17 cycles, preferably 14-16 cycles, or the dissociation constant of the enriched aptamer pool reaches below 1 μM, where in the oligonucleotides of (a), (b), (c) and (d) are DNA, RNA, DNA with modified nucleotides listed in Table 1, and/or RNA with modified nucleotides listed in Table 1.

In addition, the randomized oligonucleotides in the two libraries range in size from about 60 to about 200 nucleotides, and comprise an internal random region and an internal fixed region (arm), wherein each random region is flanked by the fixed region. The fixed region comprises at least one primer region comprising an oligonucleotide tag on the respective 5′- and 3′-termini of the A and B oligonucleotides, and an oligonucleotide tag of 4-6 fixed nucleotides.

In one aspect, the affinity based partitioning is a Systematic Evolution of Ligands by Exponential Enrichment (SELEX), or any variation of SELEX. Generally, the target of interest is selected from the group consisting of a peptide, a protein, a nucleic acid, a cell, a component of living tissue, an organic molecule, and an inorganic molecule.

In a more particular embodiment, the invention provides a method for isolating pairs of oligonucleotide aptamers for selective binding to a target of interest, starting with RNA oligonucleotides comprising:

(a) preparing two libraries of randomized RNA oligonucleotides ranging in size from about 60 to about 200 nucleotides;

(b) independently screening each library of (a) by affinity based partitioning against the target of interest, to obtain respective A and B pools of RNA oligonucleotides enriched with RNA oligonucleotides that bind to the target of interest;

(c) incubating the pool A and pool B RNA oligonucleotides with the target of interest, and a connector oligonucleotide, in order to form a four part complex of two oligonucleotides, the connector and the target of interest, wherein the connector oligonucleotide that keeps each randomized region in two oligonucleotides in distance between about 40 and about 200 nucleotides;

(d) adding a ligase enzyme to the incubated complex of (c) to form a covalent linkage between an RNA oligonucleotide from pool A and an RNA oligonucleotide from pool B, as bound to the target;

(e) amplifying the ligated RNA oligonucleotide of (d) by a reverse transcription-polymerase chain reaction (RT-PCR) to produce a DNA oligonucleotide encoding two RNA aptamers;

(f) amplifying the DNA oligonucleotide of (e) with primers selected to separate DNA oligonucleotides encoding an RNA aptamer of pool A (aptamer A) and an RNA aptamer from pool B (aptamer B); and

(g) subjecting the DNA oligonucleotides of (f) to in vitro transcription to produce RNA oligonucleotide aptamer pairs,

wherein the oligonucleotides of each respective library comprise an internal random region, and an internal fixed region (arm), wherein each random region is flanked by the fixed region. The fixed region comprises at least one primer region comprising an oligonucleotide tag on the respective 5′- and 3′-termini of the A and B oligonucleotides, and an oligonucleotide tag of 4-6 fixed nucleotides.

In a further aspect, the primers are at least 15 nucleotides in length, and preferably about 20 nucleotides in length.

The method further includes amplifying the ligated RNA oligonucleotide by a reverse transcription-polymerase chain reaction (RT-PCR) to produce a DNA oligonucleotide encoding two RNA aptamers,

In an additional aspect, the step of in vitro transcription to produce RNA oligonucleotide aptamers is optionally conducted after introducing a suitable promoter 5′ to two double-stranded DNA oligonucleotides encoding RNA aptamers. The promoter is, for example, a T7 promoter.

In an additional aspect, the method further includes adding, after step (c), adapters or primer duplexes in order to extend the fixed oligonucleotides in pools, wherein the adapters or primer duplexes are two hybridized oligonucleotides comprising primers.

In an additional aspect, the method further includes adding, after step (f), hydrolyzing a part of pool B under alkaline conditions.

In a more specific embodiment the invention provides a method for isolating pairs of oligonucleotide aptamers for selective binding to a target of interest, the method comprising:

(a) preparing two libraries of randomized RNA oligonucleotides ranging in size from about 60 to about 200 nucleotides;

(b) independently screening each library of (a) by affinity based partitioning against the target of interest, to obtain respective A and B pools of RNA oligonucleotides enriched with RNA oligonucleotides that bind to the target of interest;

(c) incubating the pool A and pool B RNA oligonucleotides with the target of interest, and a connector oligonucleotide, in order to form a four part complex of two oligonucleotides, the connector and the target of interest, wherein the connector oligonucleotide that keeps each randomized region in two oligonucleotides in distance between about 40 and about 200 nucleotides;

(d) adding adapters or primer duplexes in order to extend the fixed oligonucleotides in pools, wherein the adapters or primer duplexes are two hybridized oligonucleotides comprising primers;

(e) adding a ligase enzyme to the incubated complex of (d) to form covalent linkages between an oligonucleotide from pool A and an oligonucleotide from pool B, as bound to the target, as well as between adapters and oligonucleotide from each pool;

(f) amplifying the ligated oligonucleotide of (e) by a reverse transcription-polymerase chain reaction (RT-PCR) to produce a DNA oligonucleotide encoding two RNA aptamers;

(g) amplifying the DNA oligonucleotide of (f) to produce two double-stranded DNA oligonucleotides encoding RNA aptamers, with primers selected to separate DNA oligonucleotides encoding an RNA aptamer of pool A (aptamer A) and an RNA aptamer from pool B (aptamer B);

(h) hydrolyzing a part of pool B in alkaline condition; and

(i) subjecting the products of (g) and (h) to in vitro transcription to produce RNA oligonucleotide aptamer enriched pool,

wherein the oligonucleotides of each respective library comprise an internal random region, and an internal fixed region (arm), wherein each random region is flanked by the fixed region. The fixed region comprises at least one primer region comprising an oligonucleotide tag on the respective 5′- and 3′-termini of the A and B oligonucleotides, and an oligonucleotide tag of 4-6 fixed nucleotides.

### BRIEF DESCRIPTION OF THE DRAWINGS

FIG. 1 illustrates a schematic diagram of RNA aptamer pair selection from two random libraries flanked by two primers.

FIGS. 2A and 2B illustrate schematic diagrams of RNA aptamer pair selection from two random libraries flanked by one primer. FIG. 2A illustrates the preparation of double stranded DNAs encoding two RNA aptamers from two sequence randomized oligonucleotides libraries. FIG. 2B illustrates the amplification and alkaline hydrolysis of DNA oligonucleotide from previous step of FIG. 2A and preparation of RNA aptamer pair.

FIGS. 3A and 3B illustrate target-dependent RNA aptamer pair pool enrichment at the first round of aptamer pair selection with a method described in FIG. 1. FIG. 3A illustrates the results with plasminogen. FIG. 3B illustrates the results with human complement 7.

FIG. 4 illustrates target-dependent RNA aptamer pair pool enrichment with 0-50 nM of human serum protein in a 1 μL sample.

FIG. 5 illustrates the sensitivity of proximity ligation assay (PLA) with aptamer pair pools as ligands, comparing a zero, second and third round aptamer pair pool with 0-2.5 nM of human serum protein in 1 μL sample.

FIGS. 6A and 6B illustrate the performance of RNA aptamer pairs identified with a method described in FIG. 2. FIG. 6A illustrates the schematic design of FRET assay to test RNA aptamer pairs. FIG. 6B illustrates the results of FRET assay.

### DETAILED DESCRIPTION OF THE INVENTION

A preferred embodiment of the invention will be described, but the invention is not limited to this embodiment. Variations and modifications can be made as well occur to those skilled in the art.

Broadly, the present invention provides for target-driven selection of RNA aptamer pairs, to be selected simultaneously as pairs of aptamers capable of selectively binding to the same target of interest. The target can be any biomaterial, biomolecule and/or other composition or material susceptible to selective binding to an aptamer, including, without limitation, a peptide, a protein, a DNA or RNA molecule, a cell, a component of living tissue, an organic molecule, and/or an inorganic molecule, toxins, viruses, bacteria.

The process is started by generating two randomized single-stranded RNA oligonucleotide libraries.

The oligonucleotide libraries can be in sizes ranging from about 60 to about 200 nucleotides including randomized RNA in sizes ranging from 20 to 100 nucleotides. Two random RNA oligonucleotide libraries flanked by different RNA sequences on respective 5′- and 3′-terminals are pre-screened by affinity based partitioning. This can be accomplished by applying the SELEX method, or the libraries can be prescreened by any other art-known affinity based method, against a target of interest. Methods for screening for aptamers are described, for example, by WO2000056930A1.

For the present invention, SELEX is only conducted through several rounds (e.g., from 1 to 6 rounds), starting with e.g., two random oligonucleotide libraries, in order to produce pools of oligonucleotide molecules enriched for molecules (pool A and pool B) capable of selectively binding to the target of interest.

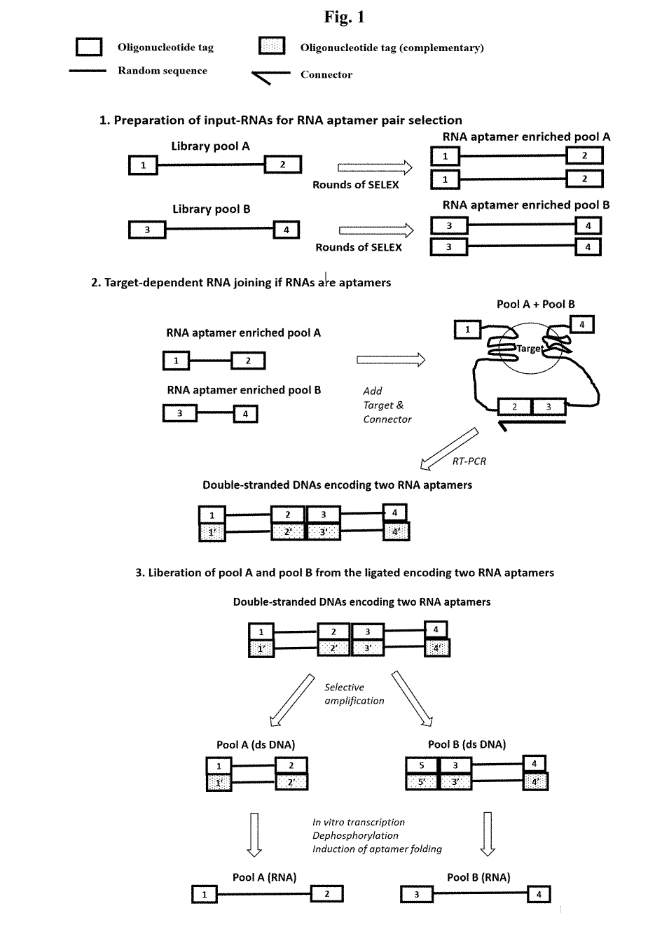

FIG. 1 shows how paired RNA oligonucleotide aptamer candidates are recruited in the presence of a target and then prepared as a pool for the next round of selection. Oligonucleotides that are cooperatively bound in a complex with the target and nucleotide connector will be preferentially “marked” for amplification (e.g., RT-PCR). A target molecule recruits an aptamer that originated in Pool A and its paired aptamer that started from Pool B.

The oligonucleotides from the pools that were enriched from libraries A and B are incubated together with the target of interest, and in the presence of a connector oligonucleotide. The connector oligonucleotide is an oligonucleotide, ranging in size from about 10 to about 50 nucleotides, or more particularly from about 18 to about 22 nucleotides in length. The connector oligonucleotide is complementary to the internal fixed region (the 3′-end of the pool A oligonucleotides and the 5′-end of the pool B oligonucleotides). The RNA aptamers from pool A and pool B can remain in proximity up to 200 nucleotides, which the corresponding length is approximately 150 nm, depending upon the target size.

### Schematic Diagram of RNA Aptamer Pair Selection From a Random Library Flanked by Two Primers (FIG. 1)

Panel 1 illustrates preparation of input RNAs for RNA aptamer pair selection;

Panel 2 illustrates target dependent joining and the following amplification by RT-PCR if the candidate RNA oligonucleotides are aptamers; and

Panel 3 illustrates liberation of pool A and pool B from the amplified ligated encoding two RNA aptamers by selective amplification, in vitro transcription, dephosphorylation and induction of aptamer folding.

The term aptamer is applied herein once the oligonucleotide has shown it will bind specifically to the target of interest. The pool A and pool B derived oligonucleotides form a three-molecule interaction (an aptamer from pool A, an aptamer from pool B, and the target) to comprise a target—aptamer pair complex that greatly enhances the hybridization energy of recruited aptamer pairs to a short oligonucleotide connector, through their pre-designed internal fixed region (arm). In particular, the 3′ tag on the pool A RNA oligonucleotide and the 5′-tag on the pool B RNA oligonucleotides are the regions that will be hybridized to the connector oligonucleotide. The 5′ tag of pool A oligonucleotides and the 3′ tags of the pool B oligonucleotides are the regions in which the primers bind for selective amplification after joining of the selected oligonucleotides from pool A and pool B, respectively, by ligation.

The advantage of obtaining pairs of ligands is that much greater levels of sensitivity and selectivity can be achieved in an assay or clinical application, by applying two different ligands targeted to different binding sites (e.g., epitopes) of a target moiety. This is a result of cooperative stabilization, or the “proximity effect.” The proximity effect results in the elevated concentration of pairs of aptamers near a connector due to target binding. This proximity enhances the hybridization energy of the two aptamer pairs that are in proximity to the short oligonucleotide connector.

When complexed with the connector oligonucleotide, a four-molecule complex (target—aptamer pair complex hybridized to oligonucleotide connector) results. The four-molecule complex is then subjected to a ligation reaction, e.g., by adding a ligase enzyme, such as an RNA or DNA ligase, to form a covalent linkage between paired aptamers, thus “marking” them for amplification. The ligated products encoding a pair of the recruited aptamers is preferentially amplified, e.g., by RT-PCR, using a pair of primers which specifically recognize pool A and pool B oligonucleotides.

This selection process results in the conversion of the target-aptamer pair complex to a proportional amount of ligated aptamers, quantitatively.

All of the above steps proceed in free solution, making physical partitioning of the pool (i.e. washing) unnecessary before amplification. The selection process is repeated for multiple rounds, i.e., reiterated, by mobilizing two aptamer pools from the ligated product pool using subsequent enzymatic reactions. These enzyme reactions do not serve as major selective pressures that change the DNA population.

Incorporation of modified nucleotides into in vitro RNA or DNA selections offer many potential advantages, such as the increased stability of selected nucleic acids against nuclease degradation, improved affinities, expanded chemical functionality, and increased library diversity. Introducing modifications with novel base pairing may potentially provide additional chemical and functional properties, unrestricted by unmodified nucleotide base pairing. Modified nucleotide pools can also potentially increase the overall binding affinities of selected aptamers. Aptamer-target binding is generally mediated by polar, hydrogen bonding, and charge-charge interactions. In contrast, hydrophobic contacts that contribute to protein-protein interactions are limited. Hence, addition of functional groups that mimic amino acids side chains may expand chemical diversity and enhance the binding affinity of aptamers.

Modifications of the ribose 2′-OH is one optional approach to increase the stability of RNA. The small electronegative 2′ substituents such as 2′-fluoro (2′-F), DNA (2′-H), 2′-O-methyl (2′-OMe) are most widely used as they are well-tolerated, generally enhance RNA nuclease resistance while not dramatically affect RNA thermostability and conformation. Fluorine substitution (2′-F) slightly stabilizes dsRNA duplexes (-1° C. increase in Tₘ per modification), is among the best tolerated modification types. Additional to 2′-F, a 2′-OMe modification is also known to be well-tolerated in the RNA structure and to increase nuclease resistance. Commonly used 2′ substituents includes ribonucleic acids possessing 2′-aminoethyl, deoxyribonucleic acid, 2′-fluoro, 2′-O-methyl, or 2′-O-methoxyethyl, and 2′-deoxy-2′-fluoro-β-D-arabinonucleic acid, 4′-C-hydroxymethyl-DNA, locked nucleic acid, 2′,4′-carbocyclic-LNA-locked nucleic acid, oxetane-LNA, unlocked nucleic acid, 4′-thioribonucleis acid, 2′-deoxy-2′-fluoro-4′-thioribonucleic acid, 2′-O-Me-4′-thioribonucleic acid, 2′-fluoro-4′-thioarabinonucleic acid, altritol nucleic acid, hexitol nucleic acid.

The oligonucleotides contemplated can optionally include a phosphorothioate internucleotide linkage modification, sugar modification, nucleic acid base modification and/or phosphate backbone modification. The oligonucleotides can contain natural phosphorodiester backbone or phosphorothioate backbone or any other modified backbone analogues, including, optionally LNA (Locked Nucleic Acid), PNA (nucleic acid with peptide backbone), CpG oligomers, and the like, such as those disclosed at the Tides 2002, Oligonucleotide and Peptide Technology Conferences, May 6-8, 2002, Las Vegas, Nev. and Oligonucleotide & Peptide Technologies, 18 & 19 Nov. 2003, Hamburg, Germany, the contents of which are incorporated herein by reference.

Oligonucleotides according to the invention can also include any suitable art-known nucleotide analogs and derivatives, such as including but not limited to those listed by Table 1, below.

TABLE 1
Representative Nucleotide Analogs and Derivatives
For Optional Substitution

| | | |
|---|---|---|
| N-((9-beta-D- | N6-isopentenyladenosine | Uridine-5-oxyacetic acid |
| ribofuranosylpurine-6-yl)- | | |
| carbamoyl)threonine | | |
| 2′-Omethyl-5-methyluridine | 1-methyl adenosine | 8-Oxoadenoosine |
| 2′-O-methyluridine | 1-methyl guanosine | Isoguanosine |
| Wybutoxine | 1-methyl inosine | 2-aminoadenosine |
| 3-(3-amino-3-carboxy- | 2,2-dimethylguanosine | 2-amino-6- |
| propyl)uridine | | chloropurineriboside |
| Locked-cytidine | 2-methylguanosine | 8-Azaadenosine |
| Locked-thymine | 2-methyladenosine | 6-chloropurineriboside |
| Locked-methylcytidine | 3-methylcytidine | 5-Iodocytidine |
| 4-acetylcytidine | 5-methylcytidine | 5-Iodouridine |
| 5-(carboxyhydroxymethyl) | N6-methyladenosine | 5-methylcytidine |
| uridine | | |
| 2′-O-methylpseudouridine | 7-methylguanosine | 5-methyluridine |
| D-galactosylqueuosine | 5-methylaminomethyluridine | 4-thiouridine |
| 2′-O-methylguanosine | Locked-adenosine | O6-methylguanosine |
| Inosine | Locked-guanosine | 2-thiouridine |
| 5-methoxyaminomethyl-2- | Nocked-uridine | 5,6-dihydrouridine |
| thiouridine | | |
| Beta.D-mannoylqueuosine | Wybutoxisine | 2-thiocytidine |
| 5-methoxyuridine | Peudouridine | 2′-Fluoro-2′-deoxycytidine |
| 2-methylthio-N6- | Queuosine | 2′-Fluoro-2′-deoxyuridine |
| isopentenyladenosine | | |
| N-((9-beta_D- | 2-thiocydidine | Deoxyuridines possessing |
| ribofuranosylpurine-6-yl)N- | | the protein-like side |
| methylcarbamoyl)threonine | | chains at the 5′-position |
| | | (FIG. 1, Molecular |
| | | Therapy - Nucleic Acids |
| | | (2014) 3, e201; |
| | | doi: 10.1038/mtna.2014.49 |
| Uridine-5-oxyacetic acid- | 5-methyl-2-thiouridine | Ds and Px (Nature |
| methylester | | Biotechnology (2013); |
| | | doi: 10.1038/nbt.2556) |

Modifications to the oligonucleotides contemplated by the invention include, for example, the addition to or substitution of selected nucleotides with functional groups or moieties that permit covalent linkage of an oligonucleotide to a desirable polymer, and/or the addition or substitution of functional moieties that incorporate additional charge, polarizability, hydrogen bonding, electrostatic interaction, and functionality to an oligonucleotide. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodouracil, backbone modifications, methylations, base-pairing combinations such as the isobases isocytidine and isoguanidine, and analogous combinations. Oligonucleotides contemplated within the scope of the present invention can also include 3′ and/or 5¹ cap structure. See examples of nucleoside analogues described in Freier & Altmann, 1997, Nucl. Acid Res.; 25, 4429-4443 and Uhlmann, 2000, Curr. Opinion in Drag Development, 3(2), 293-213.

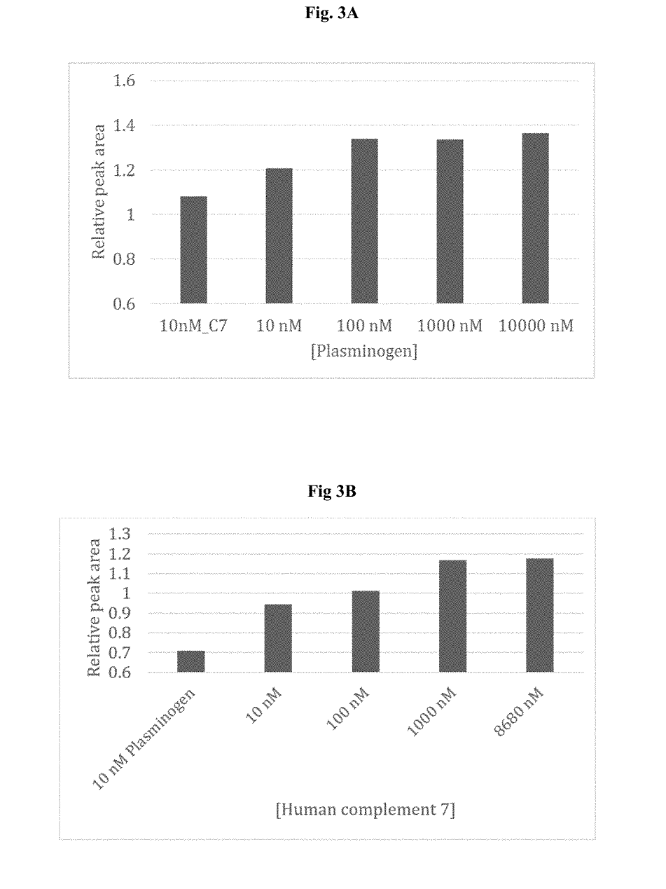

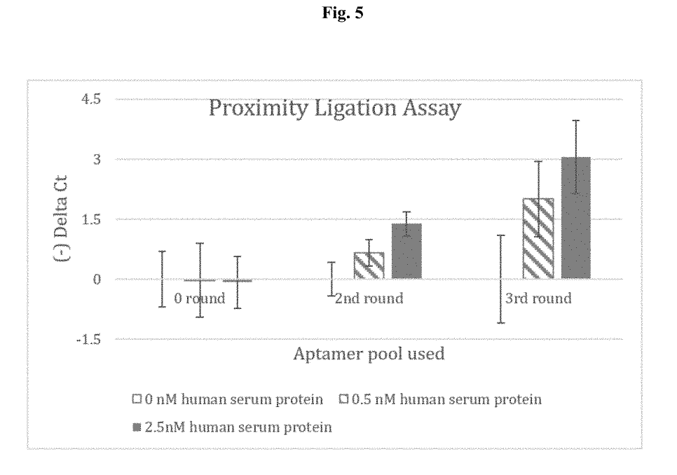

Without being bound to any theory, several advantages of the present invention include:
The inventive RNA aptamer pair selection does not require a characterized aptamer pool to find aptamer pairs. As long as aptamers having two distinct target binding sites are available in two pools, the present RNA aptamer pair selection yields aptamers in one pool and the paired aptamers in the other pool. This allows a homogeneous selection of aptamer pairs in which characterization of aptamers is inherited.
The selection process is easily monitored at each round through the amount of amplified ligated products produced in the presence/absence of the target, i.e. continuous, quantitative assessment (see FIGS. 3A and 3B, 4 and 5).
Unpurified, unsequenced pools of aptamers can be immediately used for sandwich assays such as proximity ligation assay (see FIG. 5), directly after the final RNA aptamer pair selection round. This provides a less expensive alternative, akin to pairs of polyclonal antibodies (albeit with higher batch-to-batch variation).
The present RNA aptamer pair selection renders many post-selection steps unnecessary (e.g. pairwise combinatorial screening with two-dimensional aptamer matrix). Pairwise matching to screen aptamer pairs is done simply by sequencing the ligated products. Selection should be completed after only a few rounds of RNA aptamer pair selection. The technique is based on the proximity effect, i.e. the entropically stabilized, cooperative evolution of aptamers from two pools in the presence of the target. Zhang, et al., 2013 (Angewandte Chemie, doi:10.1002/anie.201210022) stated that DNA assembly in proximity assays in the presence of a target results in a ˜4×10⁵ fold increase in local concentration of the two probes. Starting with this number, if it is supposed there are several hundred aptamers in 10¹² random sequences, then the molar ratio of aptamer pairs to random sequences would reach to 1:1 after only two rounds of RNA aptamer pair selection. In another study, Liu et al., in 2014 (Journal of the American Chemical Society, doi:10.1021/ja412934t), estimated that five aptamers ranging in binding affinity from K_{d}=0.2 nM to 3.2 μM would be present out of 67,858 possible combinations. Starting from this assumption on abundance, the required number of RNA aptamer pair selection rounds to find aptamer pairs is at most three.

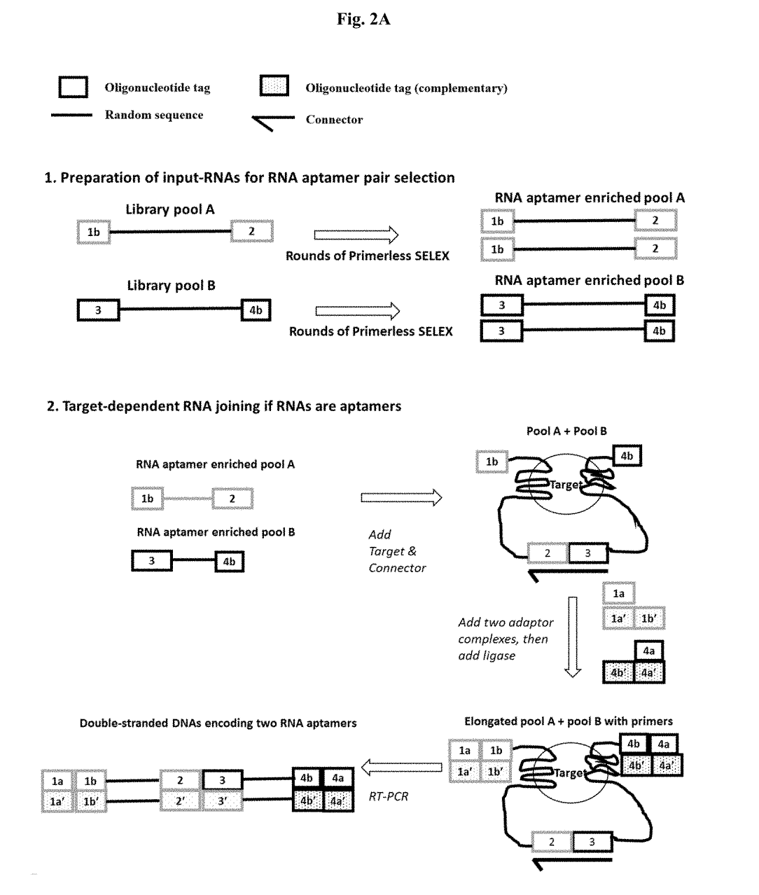

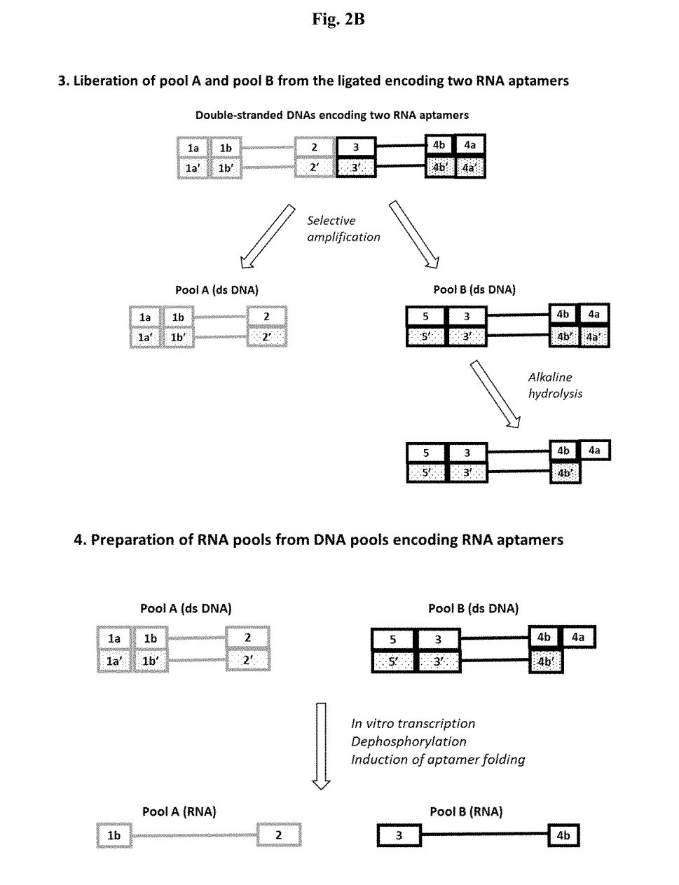

The aptamer pair selection scheme shown in Schematic diagram of RNA aptamer pair selection from random libraries flanked by one primer (FIGS. 2A and 2B) allows minimizing the participation of fixed sequences during selection so that the selected aptamers are short in length and have more flexibility in modification. Aptamers identified through standard SELEX process usually comprise 60-120 nucleotides: 30-70 nucleotide long randomized regions plus fixed primer sites of ˜15-25 nucleotide on each side. However, truncation SELEX requires about 4-6 fixed nucleotides on each side of the 30-40 randomized nucleotide sequences, making post-selection steps be simplified. This truncation SELEX technology adapted in our aptamer pair selection platform is described in WO2000056930A1. Our RNA library consists of a randomized region that is flanked by six nucleotides long stretches of fixed sequence on 5′-end of library A (1b) and 3′-end of library B (4b). As it is, it is not enough long to serve as primers for the successive amplification. After selection, they serve as hybridization sites for the bridging oligonucleotides in the pre-annealed double-stranded adapters (1a-1a′-1b′ and 4a-4a′-4b′). After ligation, the ligate in pool A (1a+1b) is not only a forward primer site but also contains a T7 promoter at its 3′ end. The ligate in pool B (4a+4b) is a reverse primer site for PCR. Uridines (U) in 4a′ allow for primer removal under alkaline condition before pool B being subjected to in vitro transcription to produce its corresponding RNA pool.

### Schematic Diagram of RNA Aptamer Pair Selection From Random Libraries Flanked by One Primer (FIGS. 2A and 2B):

Panel 1 of FIG. 2A illustrates preparation of input RNAs for RNA aptamer selection.

Panel 2 of FIG. 2A illustrates target dependent aptamer joining if RNA are aptamers.

Panel 3 of FIG. 2B illustrates liberation of Pool A and Pool B from the ligated encoding two RNA aptamers by selective amplification and alkaline hydrolysis of pool B.

Panel 4 of FIG. 2B illustrates preparation of RNA pools from DNA pools encoding RNA aptamers.

### EXAMPLES

Selected embodiments of the invention will be described in further detail with reference to the following experimental and comparative examples. These examples are for illustrative purposes only and are not intended to limit the scope of the invention.

### Example 1

### Preparation of Random RNA Library to be Used for SELEX

Two single strand DNA libraries (i.e. Library A and Library B) containing randomized sequences flanking by different tags on both 5′ and 3′-end were converted to double strand DNA libraries and amplified, by three rounds of polymerase chain reaction. The amplified double stranded DNA libraries were subjected to in vitro transcription in the mixture of four different kinds of deoxynucleoside triphosphates (i.e. dATP, dGTP, 2′-Fluoro-2′-deoxycytidine-5′-triphosphate, 2′-Fluoro-2′-deoxyuridine-5′-triphosphate) to produce the corresponding RNA libraries. RNAs in each library were then dephosphorylated on their 5′-end using 5′-RNA polyphosphatase. The input pools for the next round of selection were prepared from these dephosphorylated RNA pools by heating at 94 ° C. for 5 min followed by cooling down to 22 ° C.

### Example 2

### Validation of RNA Aptamer Pair Selection With Three Human Serum Proteins

Per target, two random libraries flanking by different tags on both 5′ and 3′-end were subjected to SELEX to enrich aptamer pools (FIGS. 3A and 3B). After 5^{th} round of SELEX with human plasminogen or complement 7, a pair of enriched aptamer pools (e.g. Pool A and Pool B) were incubated with different amounts of target at the first round of RNA aptamer selection. Subsequent ligation and amplification allow the ligated product which a pair of RNA aptamers are physically linked can be identified by gel electrophoresis.

In FIGS. 3A and 3B, peak area in an electropherogram (plot of results from electrophoresis separation) represents the amount of DNA oligonucleotide which two RNA oligonucleotides from Pool A and B are covalently linked each other and subsequently reverse-transcribed and amplified. Therefore, target-dependent increase in peak area indicates that target protein recruits more RNA oligonucleotides nearby during the first RNA aptamer pair selection. Considering a proximity effect of probes by target protein in Proximity Ligation Assay, we rationalize that this recruitment is fairly easily done if RNA oligonucleotides are a pair of aptamers. RNA aptamer pair enrichment by human plasminogen (FIG. 3A) and by human complement 7 (FIG. 3B) are shown.

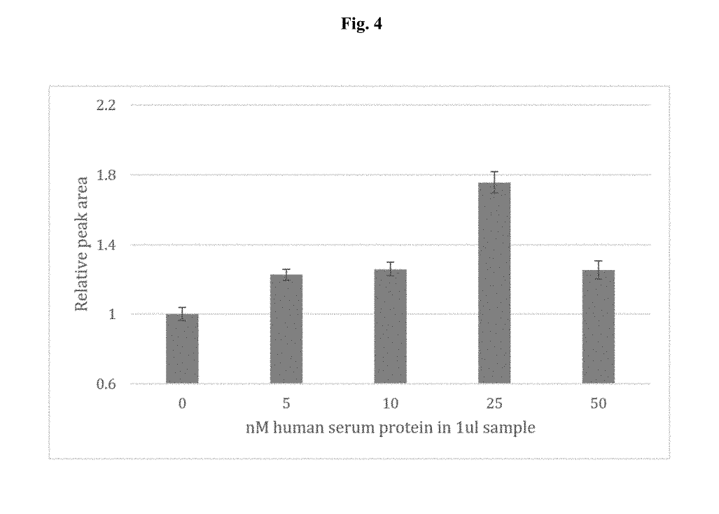

Aptamer enrichment with another serum protein was done using truncated SELEX shown in FIGS. 2A and 2B. After two rounds of aptamer enrichment, Pool A and B were subjected to aptamer pair selection. FIG. 4 shows how a pair of pool respond to target protein by representing the amount of DNA oligonucleotide encoding two RNA oligonucleotides, a result of RNA joining. The human serum protein enhanced RNA joining, thus increasing the amount of amplified ligated double strand DNA up to 1.8-fold with 25 nM protein. The reduced amount of the amplified ligated with 50 nM protein indicates that excess amount of target proteins has a negative impact on target-driven aptamer joining.

FIG. 5 shows the sensitivity of PLAs performed with three different aptamer pair pools as probes, (1) pool A and B not being subjected to aptamer pair selection, (2) Pool A and B enriched by the 2′ round of aptamer pair selection, and (3) pool A and B enriched by the 3^{rd} round of aptamer pair selection. Aptamer pools not being subjected to aptamer pair selection did not respond to human serum protein at all. Aptamer pools enriched by the 3^{rd} round aptamer pair selection responded to human serum protein more sensitively than those by the 2^{nd} round aptamer pair selection did. It represents the paired aptamers are further enriched as the round of selection goes.

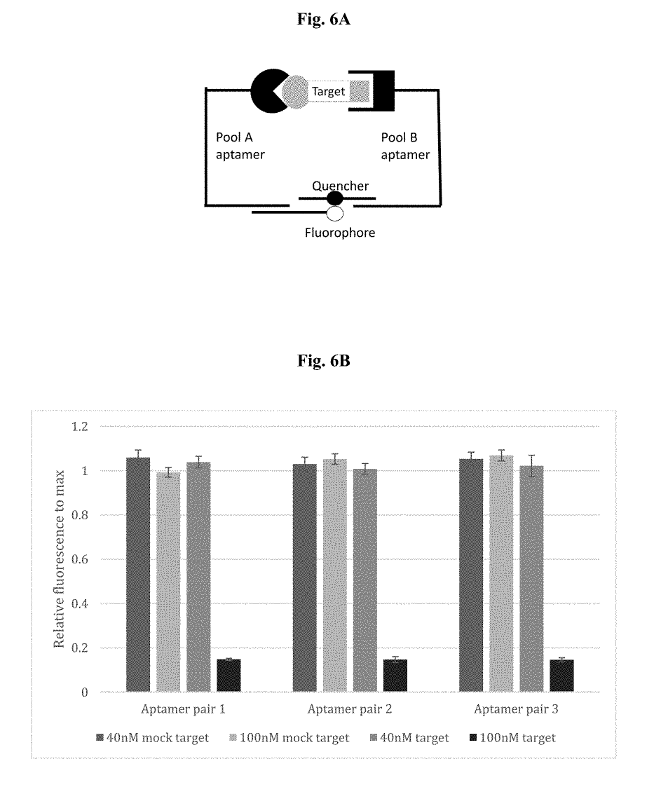

FIG. 6A shows the schematic of the designed FRET assay to test a pair of aptamers identified. The DNA strand labeled with a fluorophore is designed to be hybridized with an aptamer identified from pool A, and the other DNA strand labeled with a quencher is designed to be hybridized with an aptamer identified from pool B. Consequently, upon a simultaneous target binding, a fluorophore and a quencher is in close proximity enough to make a fluorescence quenching. FIG. 6B shows assay performance of three different FRET assay. All three FRET assay respond to 100 nM target by showing 85% fluorescence quenching. While none of assay does not respond to 100 nM mock target, showing the exclusive binding of aptamer pairs to the corresponding target.

### Example 3

### Use the Present Invention to Optimize Aptamer Pair Selection Platform in the Presence of a Given Target

Monitoring of aptamer pair enrichment in each round of selection makes it possible to adjust selective pressure applied to the next round of selection. For instance, selective pressure would be kept constant in the round of selection until a substantial increase in the amount of ligated product being observed. Then, a pair of pool in the following selection would be exposed to much stringent condition such as low amount of a target, to allow the enriched aptamers in one pool competing each other to find their pairs in the other pool. Eventually, this would lead to shorten the number of selection rounds to obtain the best aptamer pairs.

The invention also enhances understanding of the initial pools for aptamer pair selection. Several different initial pools (e.g. 3^{rd} 5^{th}, and 7^{th} round aptamer-enriched pools) are subjected in parallel with a given amount of target protein, expecting high success rate in finding aptamer pairs. For example, if only the 3^{rd} round pool yields a positive result in finding aptamer pairs, then the negative results (e.g. 5^{th} and 7^{th} round aptamer-enriched pools) are interpreted as being aptamer pairs that were outcompeted by individual aptamers. If only the 7^{th} round pool yields a positive result, then additional the aptamer pair selection method rounds may give positive results in both 3^{rd} and 5^{th} round aptamer-enriched pools. This type of data does not give any consensus in the number of aptamer-enriching cycles required in the initial pool, but the data helps to greatly reduce systematic errors, such as PCR artifacts, by suggesting the maximum allowed PCR cycles in individual aptamer-enriching steps.

Aptamer-enriched pools are initially evaluated using the proximity ligation assays (PLA), which is highly compatible with the present aptamer pair selection scheme. Patent publications disclosing the PLA that is used as a platform for aptamer pair selection in our technology are as follows. U.S. Pat. No. 7,306,904 B2 is the first filed patent for the PLA. US20080293051A1 teaches a PLA with RNAs as probes. The assay is the same as that disclosed by U.S. Pat. No. 7,306,904B2, except that RNAs are used as probes, and RNA ligase is used instead of DNA ligase. A publication disclosing truncation SELEX to minimize the participation of fixed sequences used in our technology is WO2000056930A1. The PLA assay is also highly sensitive, commonly exhibiting limits of detection (LODs) in the low attomole range.

Then, screening for the PLA dynamic range of the aptamer-enriched pools is performed. Protein amounts are varied over multiple orders-of-magnitude, from 1 amol through 1 nmol (10⁻¹⁸ through 10⁻⁹ mol) while evaluating PLA response (qPCR readout). After the approximate dynamic range of the assay is determined, the assay range is further refined, and PLA is carried out over this narrowed range (at least 10 different protein concentrations). Ultimately, performance metrics such as LOD, LOQ, dynamic range, and sensitivity are measured.

The selected pairs would be evolved cooperatively during the rounds of selection, so that it is expected that identification of aptamer pairs from the pools is done by sequencing of the ligated double strand DNAs. The resulting RNA from the ligated double strand DNA can be a new ligand enhancing its affinity and specificity inherited from two RNA aptamers in case of its folding mechanism not being affected by covalent linking of aptamers.

### INCORPORATION BY REFERENCE

Numerous publications are cited hereinabove, all of which are incorporated by reference herein in their entireties.

1. A method for isolating pairs of oligonucleotide aptamers for selective binding to a target of interest, the method comprising:
(a) preparing two libraries of sequence randomized oligonucleotides;
(b) independently screening each library of (a) by affinity based partitioning against the target of interest, to obtain respective A and B pools of oligonucleotides enriched with oligonucleotides that bind to the target of interest;
(c) incubating the pool A and pool B oligonucleotides with the target of interest, and a connector oligonucleotide, in order to form a four part complex of two oligonucleotides, the connector and the target of interest;
(d) adding a ligating enzyme to the product of (c) to ligate the oligonucleotides of the complex to form a ligated oligonucleotide; and
(e) amplifying the ligated oligonucleotide of (d) by a polymerase chain reaction (PCR) or by a reverse transcription polymerase chain reaction (RT-PCR) to produce a DNA oligonucleotide encoding two oligonucleotide aptamers.

2. The method of claim 1, further comprising subjecting the oligonucleotide pairs to one or more additional cycles of enrichment by repeating steps (c) through (e) until the desirable affinity and specificity of the obtained oligonucleotide aptamer pairs has been achieved.

3. The method of claim 1, wherein the oligonucleotides of (a), (b), (c) and (d) are DNA.

4. The method of claim 1, wherein the oligonucleotides of (a), (b), (c) and (d) are RNA.

5. The method of claim 1, wherein the oligonucleotides of (a), (b), (c) and (d) are DNA containing modified nucleotides listed in Table 1.

6. The method of claim 1, wherein the oligonucleotides of (a), (b), (c), and (d) are RNA containing modified nucleotides listed in Table 1.

7. The method of claim 1, wherein the randomized oligonucleotides range in size from about 60 to about 200 nucleotides, and comprise an internal random region and an internal fixed region, wherein each internal random region flanked by internal fixed regions comprising independently selected oligonucleotide tags on the respective 5′- and 3′-termini of the A and B oligonucleotides.

8. The method of claim 1, wherein the affinity based partitioning is Systematic Evolution of Ligands by Exponential Enrichment (SELEX) or any variation of SELEX.

9. The method of claim 1, wherein the target of interest is selected from the group consisting of a peptide, a protein, a nucleic acid, a cell, a component of living tissue, an organic molecule, and an inorganic molecule.

10. The method of claim 1, the method comprising:
(a) preparing two libraries of randomized RNA oligonucleotides ranging in size from about 60 to about 200 nucleotides;
(b) independently screening each library of (a) by affinity based partitioning against the target of interest, to obtain respective A and B pools of RNA oligonucleotides enriched with RNA oligonucleotides that bind to the target of interest;
(c) incubating the pool A and pool B RNA oligonucleotides with the target of interest, and a connector oligonucleotide, in order to form a four part complex of two oligonucleotides, the connector and the target of interest, wherein the connector oligonucleotide that keeps each randomized region in two oligonucleotides in distance between about 40 and about 200 nucleotides;
(d) adding a ligase enzyme to the incubated complex of (c) to form a covalent linkage between an RNA oligonucleotide from pool A and an RNA oligonucleotide from pool B, as bound to the target;
(e) amplifying the ligated RNA oligonucleotide of (d) by a reverse transcription-polymerase chain reaction (RT-PCR) to produce a DNA oligonucleotide encoding two RNA aptamers;
(f) amplifying the DNA oligonucleotide of (e) with primers selected to separate DNA oligonucleotides encoding an RNA aptamer of pool A (aptamer A) and an RNA aptamer from pool B (aptamer B); and
(g) subjecting the DNA oligonucleotides of (f) to in vitro transcription to produce RNA oligonucleotide aptamer pairs after introducing a suitable promoter to 5′ end of two double-stranded DNA oligonucleotides encoding RNA aptamers,
wherein the oligonucleotides of each respective library comprise an internal random region and an internal fixed region, wherein each random region flanked by internal fixed regions comprising independently selected oligonucleotide tags on the respective 5′- and 3′-termini of the A and B oligonucleotides.

11. The method of claim 10, wherein the primers are at least 15 nucleotides in length.

12. The method of claim 10, wherein the primers are about 20 nucleotides in length.

13. The method of claim 10, wherein the affinity based partitioning is Systematic Evolution of Ligands by Exponential Enrichment (SELEX) or any variation of SELEX.

14. The method of claim 10, wherein the target of interest is selected from the group consisting of a peptide, a protein, a nucleic acid, a cell, a component of living tissue, an organic molecule, and an inorganic molecule.

15. The method of claim 10, wherein the promoter is a T7 promoter.

16. The method of claim 1, the method comprising:
(a) preparing two libraries of randomized RNA oligonucleotides ranging in size from about 60 to about 200 nucleotides;
(b) independently screening each library of (a) by affinity based partitioning against the target of interest, to obtain respective A and B pools of RNA oligonucleotides enriched with RNA oligonucleotides that bind to the target of interest;
(c) incubating the pool A and pool B RNA oligonucleotides with the target of interest, and a connector oligonucleotide, in order to form a four part complex of two oligonucleotides, the connector and the target of interest, wherein the connector oligonucleotide that keeps each randomized region in two oligonucleotides in distance between about 40 and about 200 nucleotides;
(d) adding adapters or primer duplexes in order to extend the fixed oligonucleotides in pools, wherein the adapters or primer duplexes are two hybridized oligonucleotides comprising primers;
(e) adding a ligase enzyme to the incubated complex of (d) to form covalent linkages between an oligonucleotide from pool A and an oligonucleotide from pool B, as bound to the target, as well as between adapters and oligonucleotide from each pool;
(f) amplifying the ligated oligonucleotide of (e) by a reverse transcription-polymerase chain reaction (RT-PCR) to produce a DNA oligonucleotide encoding two RNA aptamers;
(g) amplifying the DNA oligonucleotide of (f) to produce two double-stranded DNA oligonucleotides encoding RNA aptamers, with primers selected to separate DNA oligonucleotides encoding an RNA aptamer of pool A (aptamer A) and an RNA aptamer from pool B (aptamer B);
(h) hydrolyzing a part of pool B in alkaline condition; and
(i) subjecting the products of (g) and (h) to in vitro transcription to produce RNA oligonucleotide aptamer enriched pool,
wherein the oligonucleotides of each respective library comprise an internal random region and an internal fixed region, wherein each internal random region flanked by at least one internal fixed region comprising a oligonucleotide tag on the respective 5′- and 3′-termini of the A and B oligonucleotides, and an oligonucleotide tag of 4-6 fixed nucleotides.

17. The method of claim 16, wherein the affinity based partitioning is Systematic Evolution of Ligands by Exponential Enrichment (SELEX) or any variation of SELEX.

18. The method of claim 16, wherein the target of interest is selected from the group consisting of a peptide, a protein, a nucleic acid, a cell, a component of living tissue, an organic molecule, and an inorganic molecule.

19. The method of claim 16, wherein the promoter is a T7 promoter.
